(19) United States
(12) Patent Application Publication
(10) Pub. No.: US 2026/0355764 A1
(43) Pub. Date: Nov. 12, 2026

(54) DEVICE FOR DISPENSING A FLUID PRODUCT

(71) Applicant: APTAR FRANCE SAS, Le Neubourg (FR)

(72) Inventor: Franck POULLAIN, La Haye Malherbe (FR)

(73) Assignee: APTAR FRANCE SAS, Le Neubourg (FR)

(21) Appl. No.: 19/137,173

(22) PCT Filed: Dec. 14, 2023
(86) PCT No.: PCT/FR2023/052017

§ 371 Date: Jun. 9, 2025

(30) Foreign Application Priority Data
Dec. 15, 2022 (FR) FR2213488

(51) Int. Cl. B05B 11/00; B05B 11/02

(52) U.S. Cl. CPC B05B 11/0027; B05B 11/02

(57) ABSTRACT

A device for dispensing a fluid product having a reservoir (10) containing fluid product and a dispensing head (20) provided with a dispensing orifice (21). The reservoir is axially movable relative to the dispensing head during actuation. A stopper/piston (50) is movable in the reservoir to dispense fluid product. The reservoir is received in an actuation body (30), the actuation body having a radial ring (35) connected to the actuation body by one or more breakable bridges (36). The radial ring cooperates with a portion of the dispensing head to form a precompression threshold that the user must overcome during actuation to break the breakable bridges and thus actuate the device. Each breakable bridge is elastically deformable to a breaking point to dampen forces exerted on the breakable bridges that are less than a predetermined threshold corresponding to the breaking point.

The present invention relates to a device for dispensing a fluid product. More specifically, the present invention relates to a unidose device for the nasal dispensing of fluid product to dispense a pharmaceutical fluid product in the nose of a user by way of one single nasal sprays.

A drawback of existing devices for nasal dispensing of fluid product is the risk of undesired actuation in the event of a fall of the device.

Documents EP0810906, U.S. Pat. No. 5,813,570, FR3116203 and US2019126303 describe prior-art devices.

An object of the present invention is to provide a device for dispensing a fluid product that does not have the above-mentioned drawbacks.

More particularly, an object of the present invention is to provide a device for dispensing a fluid product that avoids any risk of undesired actuation, for example in the event of a fall of the device.

Another object of the present invention is to provide such a device that is simple and inexpensive to manufacture and to assemble.

The present invention thus provides a device for dispensing a fluid product comprising a reservoir containing fluid product and a dispensing head that is provided with a dispensing orifice, said reservoir being axially movable relative to said dispensing head during actuation, a stopper/piston movable in said reservoir so as to dispense fluid product through said dispensing orifice during actuation, said reservoir being received in an actuation body, said actuation body comprising a radial ring connected to said actuation body by one or more breakable bridges, said radial ring cooperating with a portion of said dispensing head so as to form a precompression threshold that the user must overcome during actuation so as to break said breakable bridges and thus actuate the device, each breakable bridge being elastically deformable to a breaking point, so as to dampen forces exerted on said breakable bridges that are less than a predetermined threshold corresponding to said breaking point.

Advantageously, each breakable bridge comprises at least one curvature, allowing said breakable bridge to be elongated by deformation of said at least one curvature.

Advantageously, each breakable bridge comprises a curvature, approximately in the shape of a V.

Advantageously, each breakable bridge has two curvatures, approximately in the shape of an S.

Advantageously, each breakable bridge is of frustoconical shape, narrowing in the direction of said actuation body.

Advantageously, each breakable bridge has a contact surface with said radial ring which is greater than the contact surface with said actuation body.

Advantageously, said reservoir is formed by a body that is hollow and blind, comprising a bottom and a single opening which, in the rest position, is sealed by said stopper/piston.

Advantageously, the device comprises a hollow insert inserted and fixed in said dispensing head, said insert supporting a piercing tip to pierce said stopper/piston and thus connect said reservoir to said dispensing orifice during actuation.

Advantageously, said piercing tip is made separately and then assembled in said insert, in particular by force-fitting or by overmoulding.

In an variant, said piercing tip is moulded integrally with said insert.

Advantageously, said reservoir contains one single dose of fluid product dispensed in one single actuation.

These and other characteristics and advantages of the present invention appear more clearly from the following detailed description, given by way of non-limiting examples, and with reference to the accompanying drawings, and in which:

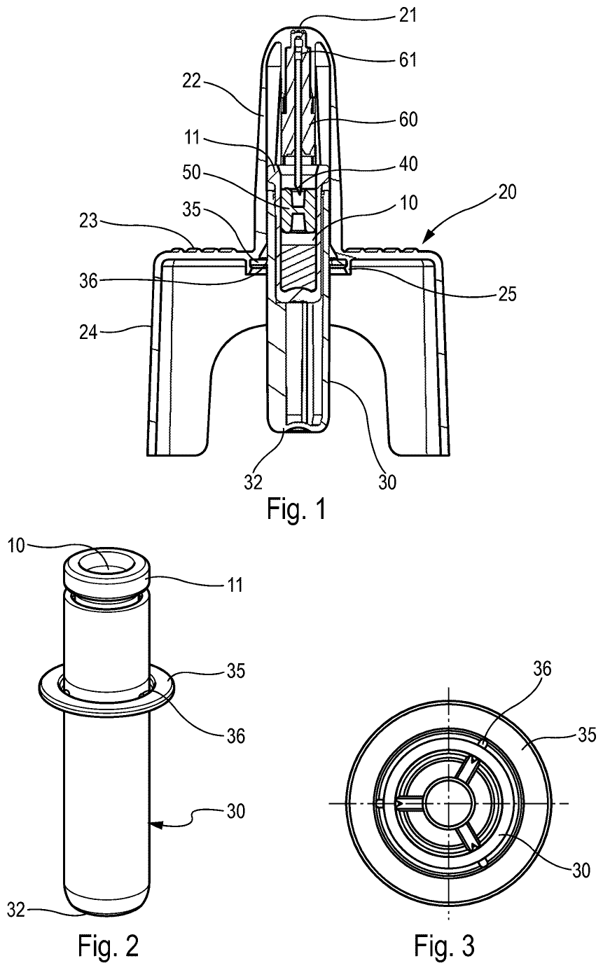

FIG. 1 is a schematic vertical cross-sectional view of a device for dispensing a fluid product according to an advantageous embodiment, in the rest position,

FIG. 2 is a schematic perspective view of an actuation body of the prior art;

FIG. 3 is a schematic horizontal cross-section view of the actuation body of FIG. 2;

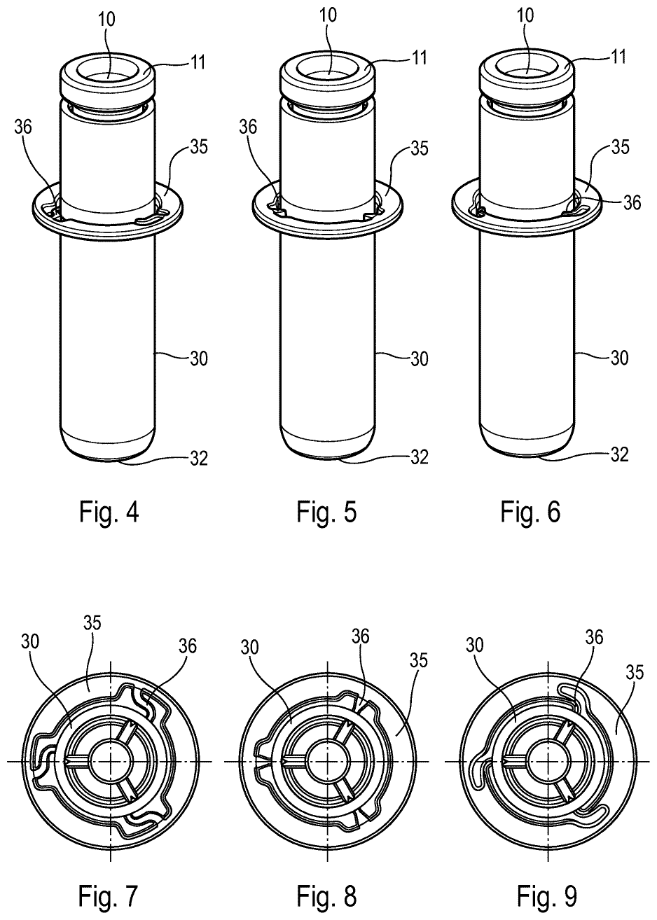

FIG. 4 is a schematic perspective view of an actuation body according to an advantageous first embodiment;

FIG. 5 is a schematic perspective view of an actuation body according to an advantageous second embodiment;

FIG. 6 is a schematic perspective view of an actuation body according to an advantageous third embodiment;

FIG. 7 is a schematic horizontal cross-section view of the actuation body of FIG. 4;

FIG. 8 is a schematic horizontal cross-section view of the actuation body of FIG. 5, and

FIG. 9 is a schematic horizontal cross-section view of the actuation body of FIG. 6.

The terms “axial” and “radial” are relative to the longitudinal central axis of the device. The terms “top”, “bottom”, “vertical” and “horizontal” refer to the upright position of the device shown in FIG. 1.

In reference to FIG. 1, a preferred embodiment of the present invention is represented. In this embodiment, the device is a unidose device, that is to say that the reservoir contains a single dose of fluid product dispensed in one single actuation.

In a known manner, a reservoir 10 containing fluid product to be dispensed, typically a liquid, is arranged inside a body that forms a dispensing head 20. The dispensing head 20 comprises a dispensing orifice 21 oriented axially. The dispensing orifice 21 serves to dispense a dose of fluid out from said dispensing head 20 while the device is being actuated by a user.

The dispensing head 20 comprises an extended nosepiece 22 comprising at its proximal axial end, said dispensing hole 21, as well as a lateral body 24, connected to said nosepiece 22 at a radial flange 23.

A hollow insert 60 is advantageously arranged in the dispensing head 20, upstream from said dispensing orifice 21, said hollow insert 60 defining a central expulsion channel and, in engagement with the bottom wall of the dispensing head 20, a spraying profile (not visible in the drawings) directly upstream from said dispensing orifice 21.

The channel 61 terminates at its bottom end in a piercing tip 40. This piercing tip 40 may be moulded integrally with the insert 60. As a variant, it could be made separately, for example out of metal or of a synthetic material other than that of the insert, and then assembled in said insert, for example by force-fitting or by overmoulding. The piercing tip 40 extends axially downwards beyond the elongated cylindrical body of the insert 60.

The reservoir 10 is advantageously formed by a blind hollow body, comprising one single opening defining a neck 11, and closed by a stopper/piston 50. In the example represented, which is a unidose, the reservoir 10 contains a single fluid product dose to dispense with one single actuation of the device. The stopper/piston 50 can be displaced in the reservoir 10 between a rest position and an actuation end position to dispense said fluid product through the dispensing orifice 21 during the actuation.

An actuation body 30 is provided so as to make it possible to actuate the device.

This actuation body 31 is movable relative to the dispensing head 20, said actuation body 31 supporting said reservoir 10 so as to move it axially relative to the dispensing head 20, towards the dispensing orifice 21. The actuation body 30 comprises a bottom wall 32.

To actuate the device, the user axially presses on the actuation body 30, to move the latter together with the reservoir 10 axially in the dispensing head 20, thus causing the piercing tip 40 to pierce the stopper/piston 50 and the dose of fluid to be dispensed through the dispensing orifice 21. During such actuation, the reservoir 10 is thus pushed axially towards the dispensing orifice 21, so that the piercing tip 40 pierces the stopper/piston 50. The contents of the reservoir 10 are thus connected to the dispensing orifice 21, and the user pressing on the actuation body 30 moves the stopper 50 in the reservoir 10 so as to dispense the fluid product.

It should be noted that the present invention could also apply to devices that do not have a piercing tip suitable for piercing a stopper/piston, as described, for example, in documents WO2019122671, WO2021069823, WO2022090675, WO2022101589, WO2022208014.

The actuation body 30 includes a radial ring 35 connected to the actuation body by one or more breakable bridges 36. This ring 35 cooperates with a portion of the dispensing head 20, in particular by snap-fitting in a snap-fitting housing 25 of the dispensing head 20, to form a precompression threshold that the user must overcome during actuation in order to break the breakable bridges 36 and thus actuate the device. This radial ring 35 also serves as a tamper proof.

FIGS. 2 and 3 show breakable bridges 36 of the prior art, which are rigid and of constant width by having a square or rectangular shape. This type of well-known breakable bridges prevents any relative movement between the parts connected by the breakable bridges, until the application of a predetermined threshold force causing the breakable bridges to break.

According to the invention, each breakable bridge 36 is elastically deformable to a breaking point, to damp forces exerted on the breakable bridges that are less than a predetermined threshold corresponding to said breaking point.

The weight and therefore the inertia of the actuation body 30 and of the reservoir 10 on the one hand or of the dispensing head 20 on the other hand are insufficient to reach the point of rupture of the breakable bridges 36 in the event of accidental fall of the device. Thus, during an accidental fall of the device, the breakable bridges 36 can deform elastically, in particular elongate, without breaking. The present invention therefore ensures perfect safety against undesired actuation in case of a fall of the device.

It is only during actuation, when the user presses simultaneously on the bottom 32 of the actuation body 30 and on the radial flange 23 of the dispensing head 20, that the breakable bridges will reach their breaking point to break and allow the dose contained in the reservoir 10 to be dispensed.

Advantageously, each breakable bridge 36 has a contact surface with said radial ring 35 which is greater than the contact surface with said actuation body 30. This ensures that the breakable bridge 36 breaks at the contact surface with the actuation body 30, to ensure reliable sliding of the actuation body 30 in the dispensing head 20 during actuation.

An advantage of the present invention is that it does not require additional movement or displacement of a portion of the device: the user simply takes the device in hand and performs the conventional actuation movement. The only difference is that the breakable bridges 36 will only break after their initial elastic deformation, when the breaking point is reached.

The advantageous embodiment shown in FIGS. 4 to 9 will now be described in greater detail below.

In the examples in FIGS. 4, 6, 7, and 9, each breakable bridge 36 has at least one curvature that allows elongation of the breakable bridge 36 by flattening each curvature. Thus, during an accidental fall, the actuation body 30 can move slightly axially relative to the dispensing head 20. This slight axial displacement, corresponding to the elongation of the breakable bridge or bridges 36, is less than the travel necessary for the needle 40 to pierce the stopper/piston 50. Thus, there is no risk of unwanted actuation during such an accidental fall.

In the example in FIGS. 4 and 7, each breakable bridge 36 comprises two curvatures, approximately in the shape of an S.

In the example in FIGS. 6 and 9, each breakable bridge 36 comprise a curvature, approximately in the shape of a V.

In the example in FIGS. 5 and 8, each breakable bridge 36 is of frustoconical shape, narrowing in the direction of the actuation body 30. In this implementation, each breakable bridge 36 may have an improved elongation or stretching capacity compared to a prior-art breakable bridge, as shown in FIGS. 2 and 3.

Naturally, other variant embodiments may also be envisaged, without going beyond the scope of the present invention, as defined by the accompanying claims.

1-11. (canceled)

12. A device for dispensing a fluid product comprising a reservoir containing fluid product and a dispensing head that is provided with a dispensing orifice, said reservoir being axially movable relative to said dispensing head during actuation, a stopper/piston being movable in said reservoir so as to dispense fluid product through said dispensing orifice during actuation, said reservoir being received in an actuation body, said actuation body comprising a radial ring connected to said actuation body by one or more breakable bridges, said radial ring cooperating with a portion of said dispensing head so as to form a precompression threshold that the user must overcome during actuation so as to break said breakable bridges and thus actuate the device, characterised in that each breakable bridge is elastically deformable to a breaking point, so as to dampen forces exerted on said breakable bridges that are less than a predetermined threshold corresponding to said breaking point, each breakable bridge comprising at least one curvature, enabling an elongation of said breakable bridge by deformation of said at least one curvature.

13. The device according to claim 12, wherein each breakable bridge comprises a curvature, approximately in the shape of a V.

14. The device according to claim 12, wherein each breakable bridge comprises two curvatures, approximately in the shape of an S.

15. The device according to claim 12, wherein each breakable bridge is of frustoconical shape, narrowing in the direction of said actuation body.

16. The device according to claim 12, wherein each breakable bridge has a contact surface with said radial ring that is greater than the contact surface with said actuation body.

17. The device according to claim 12, wherein said reservoir is formed by a hollow and blind body, comprising a bottom and a single opening which, in the rest position, is sealed by said stopper/piston.

18. The device according to claim 12, comprising a hollow insert inserted and fixed in said dispensing head, said insert supporting a piercing tip to pierce said stopper/piston and thus connect said reservoir to said dispensing orifice during actuation.

19. The device according to claim 18, wherein said piercing tip is made separately and then assembled in said insert, in particular by force-fitting or by overmoulding.

20. The device according to claim 18, wherein said piercing tip is moulded integrally with said insert.

21. The device according to claim 12, wherein said reservoir contains a single fluid product dose dispensed in a single actuation.
